(19) United States
(12) Patent Application Publication
(10) Pub. No.: US 2005/0043558 A1
(43) Pub. Date: Feb. 24, 2005

(54) Thiols and disulphides and their use in producing substrates

(76) Inventors: Luc Lebeau, Erstein-Krafft (FR); Emmanuel Klein, Niedernai (FR)

Correspondence Address: BIRCH STEWART KOLASCH & BIRCH, PO BOX 747, FALLS CHURCH, VA, 22040-0747, US

(21) Appl. No.: 10/493,822

(22) PCT Filed: Nov. 6, 2002
(86) PCT No.: PCT/EP02/12397

(30) Foreign Application Priority Data
Nov. 6, 2001 (DE) 101 53 954.1

(51) Int. Cl. B32B 9/04; C07C 335/00

(52) U.S. Cl. 558/17; 558/20; 564/17; 427/445

(57) ABSTRACT

The present invention concerns new methods for producing substrates with surfaces, that are covalently linked to at least one type of hapten or biological macromolecule, with reduced non-specific biological macromolecule adsorption. In another aspect, the invention concerns substrates with surfaces that are covalently linked to at least one type of hapten, showing circulating compounds and the use of these substrates for the quantitative detection of at least one type of hapten-specific biological macromolecule receptor, in particular a hapten-specific antibody, in a test probe. Further, the invention concerns substrates with surfaces that are covalently linked to at least one type of biological macromolecule, in particular one type of antibody, showing reduced non-specific interactions with circulating compounds and the use of these substrates for the quantitative detection of at least one type of hapten in a test probe. In another aspect, the invention is related to specific thiols of formula (A) and disulphides of formula (B), where the variables are as defined in the claims, which can be used for producing the above-mentioned substrate surfaces. The invention is further related to the use of these specific thiols and disulfides for producing substrates with surfaces that arc covalently linked to at least one type of hapten or to at least one type of biological macromolecule, showing reduced non-specific interactions with circulating compounds.

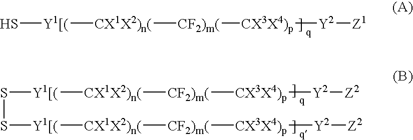

The present invention concerns new methods for producing substrates with surfaces that are covalently linked to at least one type of hapten or biological macromolecule with reduced non-specific biological macromolecule adsorption.

In another aspect, the invention concerns substrates with surfaces that are covalently linked to at least one type of hapten showing reduced non-specific interactions with circulating compounds and the use of these substrates for the quantitative detection of at least one type of hapten-specific biological macromolecule receptor, in particular a hapten-specific antibody, in a test probe.

Further, the invention concerns substrates with surfaces that are covalently linked to at least one type of biological macromolecule, in particular one type of antibody or antibody fragment, showing reduced non-specific interactions with circulating compounds and the use of these substrates for the quantitative detection of at least one type of hapten in a test probe.

In another aspect, the invention is related to specific thiols and disulfides which can be used for producing the above-mentioned substrate surfaces. The invention is further related to the use of these specific thiols and disulfides for producing substrates with surfaces that are covalently linked to at least one type of hapten or to at least one type of biological macromolecule, showing reduced non-specific interactions with circulating compounds.

Many applications involve protein specific immobilization onto a functionalized support, as for instance ELISA, SPIE-IA and a number of other format assays in biological sciences, in particular BIAcore™ technology, heterogeneous catalysis using immobilized enzymes, affinity chromatography.

For instance in DE 693 29 536 a chemically adsorbed film layer is disclosed, which comprises anchor molecules (deut. Stammmoleküle) and matrix molecules (deut. Pfropfmoleküle), wherein the anchor molecules are covalently linked to active hydrogen atoms or alkali metal atoms of the surface of the substrate using at least one element selected from the group consisting of Si, Ge, Sn, Ti, Zr, S or C, and wherein the matrix molecules are covalently linked to the at least one element by forming a bond selected from the group consisting of —SiO—, —GeO—, —SnO—, —SnN—, —TiO—, —ZrO—, —SO₂— and —C— bonds. These film layers show an increased molecule density on the substrate surface which results in an increased sensitivity of the measured signal.

In DE 199 53 667 a coated substrate is disclosed that is produced by plasmadeposition on a substrate and that is functionalized by the elongation of monomers at reactive centres on the substrate surface.

However one of the major problems met in these technologies is related to the non-specific adsorption of biological macromolecules that are present in most test probes as in particular antibodies, receptors, nucleic acids onto the support or substrate surface. In most of the cases non-specific interactions of the substrate surfaces with circulating compounds of the test probes considerably increase the background noise that is obtained using a device involving such surface, and subsequently lowers the sensitivity of the assay, the resolution power of the affinity column, or the efficacy of the enzymatic catalyst. That adsorption results from electrostatic or hydrophobic interactions, or both, between the biological macromolecule and the substrate surface.

Biological macromolecules generally comprise complex assemblies of elementary hydrophilic or hydrophobic elements as amino acids, nucleic acids, sugars, fatty acids, etc. that are combined in various manners either through covalent, or non-covalent associations. These complexes and parts of complexes thus mainly reflect the mean physical properties of their elementary building blocks in terms of water solubility, hydrophobicity, polarization and electrical charge. Consequently any biological macromolecule and any part of it may display a whole range of physical behaviors from fully hydrophilic (positively charged, negatively charged, or neutral) to hydrophobic and water insoluble. That finally results in a generally large number of possible interactions between the biological macromolecule and interfaces. These isolated interactions are generally of weak magnitude and can be easily broken. Nevertheless the combination of sets of interactions most of the time are conducing to interactions tight enough to spoil a specific immobilization process.

Different strategies have been employed to partly reduce such unwanted interactions, essentially those using polyethylene oxide (PEO) or polyethylene glycol (PEG) or dextran coatings. It is assumed that PEO and polysaccharides act in a way as a mimicry of water molecules thus preventing a close contact between the macromolecule and the support through aspecific interactions; in these cases water molecules interacting with the biological macromolecule are exchanged by PEO or polysaccharide molecules interacting with the biological macromolecules by an equilibrium process. Using such tools however, the non-specific adsorption of a biological macromolecule at an interface cannot be reduced practically down to zero. That is definitely due to the cooperativity of the interactions between the biological macromolecule and the polymer coating. Moreover the coating thickness of the support is significant and the further covalent functionalization of the surface of the support with any molecule of interest (i.e. aglycone, biological molecule) may lead to a random distribution of the later along the z axis. That introduces accessibility problem for analytes in assays for example, as well as random orientation of the trapped analytes and thus contributes to an increase of the background noise.

It is therefore the task of the present invention to provide substrates with surfaces that are funcionalized by covalent linkage to at least one biological macromolecule or to at least one hapten, showing reduced non-specific interactions with circulating compounds of the test probes and/or showing reduced non-specific adsorptions of circulating compounds of the test probes.

Further, it is the task of the present invention to provide a method for the production of those substrates and to provide means, in particular chemical compounds, that can be used for the manufacture of those substrates.

This task is solved by the provision of specific perfluorinated thiols and perfluorinated disulfides that can be used for the manufacture of substrate surfaces showing reduced non-specific interactions with circulating compounds of the test probes and/or showing reduced non-specific adsorptions of circulating compounds of the test probes and by the provision of a method to produce substrates surfaces that are funcionalized by covalent linkage to at least one biological macromolecule or to at least one hapten, showing reduced non-specific interactions with circulating compounds of the test probes.

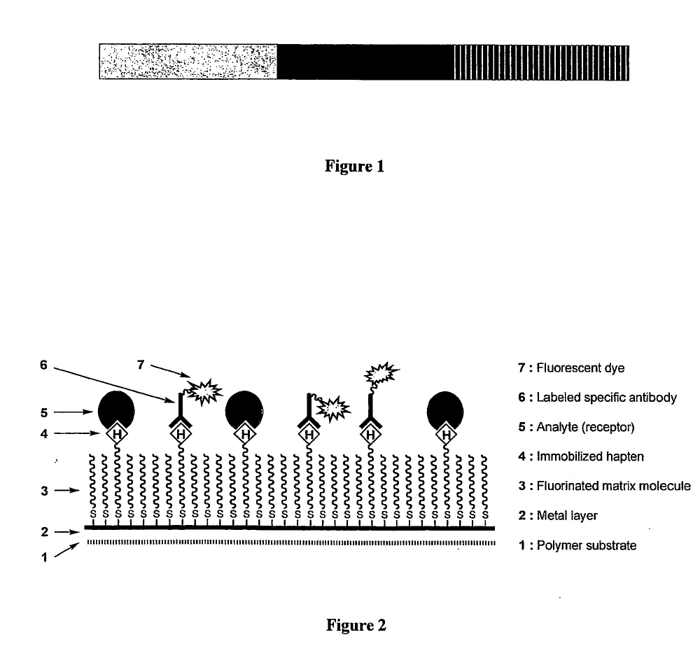

The invention makes use of the unusual properties of perfluorinated compounds for reducing aspecific binding of biological macromolecules to substrate surfaces. Perfluorinated substances are known to be chemically inert and to segregate from both hydrophilic and hydrophobic media. Perfluorinated compounds exclusively accommodate cohesive interactions with fluorinated molecules and repulsive ones with hydrophilic as well as hydrophobic (hydrocarbon) species. Therefore locally perfluorinated molecules can be used to specifically coat surfaces that have to be brought into contact with biological macromolecules for any given application in which non-specific interactions are injurious. The general architecture of the used perfluorinated compounds is formally described in FIG. 1.

The structure of the used perfluorinated compounds consists in a central linear perfluorinated chain (black segment of FIG. 1) that can be branched or not. That part plays the role of a shield to prevent macromolecules to interact with the support. At one extremity, an “anchor” (grey part of FIG. 1) is designed to interact tightly with the interface or support and protect it against aspecific interactions with biological macromolecules. The anchor can establish either a non covalent bonding with the interface (van der Waals forces, hydrophobic interaction, coulombic interaction) or a covalent one (through a chemical reaction between a reactive function at the anchor and some components at the interface). At the other extremity, a hydrophilic head group (striped part of FIG. 1) is placed to ensure the adequate wettability of the subsequent coating. These compounds correspond either fo locally perfluorinated thiols of formula (A) or to locally perfluorinated disulfides of formula (B).

In the following, underlined parts of the chemical formulas characterize cyclic parts of the structures.

The invention therefore provides chemical compounds with the formula (A):
HS—Y¹[—CX¹X²)ₙ—(CF₂)ₘ—(CX³X⁴)ₚ—]_{q}Y²—Z¹ (A)
wherein each
X¹, X², X³ and X⁴ is independently from each other selected from the group consisting of a hydrogen atom, a halogen atom, an alkyl group optionally substituted by one or several halogens, an acyl group optionally substituted by one or several halogens, a hydrocarbon group incorporating one or several double bonds optionally substituted by one or several halogens, an aralkyl group optionally substituted by one or several halogens, an aryl group optionally substituted by one or several halogens, a hydrocarbon group containing one or several heteroatoms that is optionally unsaturated;
   wherein each
Y¹ and Y² is independently from each other selected from the group consisting of
   an alkylene group optionally containing one or several heteroatoms, an alkylene group that is optionally unsaturated, an alkylene group containing one or several heteroatoms that is optionally unsaturated; preferably Y¹ is selected from the group consisting of —CH₂[—O—C₂H₄—]ₜ with t representing an integer between 0 and 10; preferably Y² is selected from the group consisting of —CH₂[—O—C₂H₄—]ᵤ with u representing an integer between 0 and 100;
      wherein each
Z¹ is selected from the group consisting of
hydrogen, halogen, a group selected from AR¹, C(B¹)(AR¹), NR¹R², ASO₂R¹, ASO₂(AR¹), SOᵣR¹, SO₂(NR¹R²), AP(B²)(AR¹)(AR²), AP(B ²)(AR²)R², P(B²)(AR¹)(AR²), P(B²)(AR¹)R², P(B²)R¹R²
wherein each A is independently from each other selected from O, S or NR¹;
each B¹ is independently from each other selected from O, S or NR¹;
each B² is selected from O, S or Se;
each R¹ and R² is selected from
hydrogen, an alkyl group optionally substituted by one or several halogens, an acyl group, an acyl group optionally substituted by one or several halogens, a hydrocarbon group incorporating one or several double bonds optionally substituted by one or several halogens, an aralkyl group optionally substituted by one ore several halogens, an aryl group optionally substituted by one or several halogens, a hydrocarbon group containing one or several heteroatoms that is optionally unsaturated, an ammonium group, an ammonium group that is substituted by at least one hydrocarbon group, an ion of formula M⁺_{1/V} in which M represents a metal and v is the valence state of metal M, an internal cation;
   and wherein
n and p independently from each other represent an integer between 0 and 4, preferably n
and p represent an integer having the value 0;
m represents an integer between 1 and 22, preferably m represents an integer between 4 and 22;
q represents an integer between 1 and 100, preferably q represents an integer having the value 1.

Further, the invention provides chemical compounds with the formula (B): 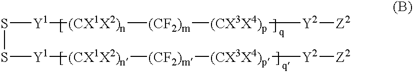
wherein each
X¹, X², X³ and X⁴ is independently from each other selected from the group consisting of hydrogen, halogen, an alkyl group optionally substituted by one or several halogens, an acyl group optionally substituted by one or several halogens, a hydrocarbon group optionally incorporating one or several double bonds, a hydrocarbon group substituted by one or several halogens and incorporating one or several double bonds, an aralkyl group optionally substituted by one or several halogens, an aryl group optionally substituted by one or several halogens, a hydrocarbon group containing one or several heteroatoms that is optionally unsaturated;
   wherein each
Y¹ and Y² is independently from each other selected from the group consisting of an alkylene group optionally containing one or several heteroatoms, an alkylene group that is optionally unsaturated, an alkylene group containing one or several heteroatoms that is optionally unsaturated; preferably each Y¹ is —CH₂[—O—C₂H₄—]ₜ with t representing an integer between 0 and 10 and preferably each Y² is selected from the group consisting of CH₂[—O—C₂H₄—]ᵤNC(O═CHCHC(O) and —CH₂[—O—C₂H₄]ₜNHC(O)CH₂Br with u representing an integer between 0 and 100;
   wherein each
Z² is independently from each other selected from the group consisting of hydrogen, halogen, a group selected from
AR¹, C(B¹)(AR¹), NRR¹, ASO₂R², ASO₂(AR¹), SOrR², SO₂(NR¹R¹), AP(B²)(AR^{1)(AR}¹), AP(B²)(AR¹)R, P(B²)(AR¹)(AR¹), P(B²)(AR¹)R, P(B²)R²R, C(B²)R, C (B²R³), a N-maleimidyl group, an isocyanate group, an isothiocyanate group, A-C(═NR¹)R², O—NHR¹;
   wherein
each A is independently from each other selected from the group consisting of O, S or NR¹;
   wherein
each B¹ is independently from each other selected from the group consisting of O, S or NR¹;
   wherein
each B² is independently from each other selected from the group consisting of O, S or Se;
   wherein
each R¹ is independently from each other selected from the group consisting of hydrogen, an alkyl group optionally substituted by one or several halogens, an acyl group optionally substituted by one or several halogens, a hydrocarbon group incorporating one or several double bonds optionally substituted by one or several halogen atoms, an aralkyl group optionally substituted by one ore several halogen atoms, an aryl group optionally substituted by one or several halogen atoms, a hydrocarbon group containing one or several heteroatoms that is optionally unsaturated, an ammonium group, an ion of formula M⁺_{1/V} in which M represents a metal and v is the valence state of metal M, an internal cation;
   wherein
each R is independently from each other selected from the group consisting of hydrogen, halogen, a N₃ group, an alkyl group optionally substituted by one or several halogens, an acyl group optionally substituted by one or several halogens, a hydrocarbon group incorporating one or several double bonds, a hydrocarbon group substituted by one or several halogen atoms optionally incorporating one or several double bonds, an aralkyl group optionally substituted by one or several halogens, an aryl group optionally substituted by one or several halogens, a hydrocarbon group containing one or several heteroatoms that is optionally unsaturated, NR¹—NHR¹;
   wherein
each R³ represents an acyl group optionally substituted by one or several halogens, SO₈R⁴ with each s independently from each other representing an integer between 0 and 2, P(B²)(AR⁴)(AR⁴), P(B²)(AR⁴)R⁴, P(B²)R⁴R⁴, C(═NR¹)(NHR¹), C═N—R⁵—NR¹, a cyclic or acyclic imide group, in particular cyclic: NC(O)R⁵C(O), or acyclic: N(C(O)R⁴)(C(O)R⁴));
   wherein
each R⁴ is independently from each other selected from the group consisting of an alkyl group optionally substituted by one or several halogens, a hydrocarbon group incorporating one or several double bonds that is optionally substituted by one or several halogens, an aralkyl group optionally substituted by one ore several halogens, an aryl group optionally substituted by one or several halogens, a hydrocarbon group containing one or several heteroatoms that is optionally unsaturated;
   wherein
each R⁵ is independently from each other selected from the group consisting of an alkylene group that optionally contains one or several heteroatoms, an alkylene group that is optionally unsaturated, a alkylene group containing one or several heteroatoms that is optionally unsaturated, an aryl diradical optionally containing one or several heteroatoms;
   wherein
each n, n′, p, and p′ independently from each other represents an integer between 0 and 4, preferably n, n′, p and p′ represent an integer having the value 0,
each m and m′ independently from each other represent an integer an integer between 1 and 22, preferably m and m′ represent an integer between 4 and 22,
each q and q′ independently from each other represent an integer between 1 and 100, preferably q and q′ represent an integer having the value 1.

In another aspect, the present invention relates to a method for producing a substrate with a surface that is linked to at least one type of hapten and that shows reduced unspecific interactions with circulating compounds that do not specifically bind to the surface-linked haptens comprising the following steps:
a) production of a metal-coated substrate by deposition of a thin metal layer onto the surface of the substrate,
b) production of a substrate that is linked to locally perfluorinated compounds by metal-sulfide bonds displaying Z¹ and/or Z² as reactive groups
   by the treatment of the metal-coated substrate of step a) with a solution comprising at least one compound selected from the group consisting of locally perfluorinated thiols with the fomula (A)
      HS—Y¹[—(CX¹X²)ₙ—(CF₂)ₘ—(CX³X⁴)ₚ—]_{q}Y²—Z¹ (A)
   which are defined as above mentioned,
   and/or comprising at least one compound selected from the group consisting of locally perfluorinated disulfides with the formula (B) 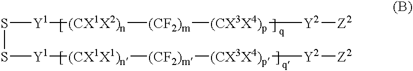
   which are defined as above mentioned,
   in an appropriate solvent, which is preferentially methanol or ethanol, for an appropriate time and at an appropriate temperature,
c) production of a substrate that is covalently linked with at least one type of hapten by the treatment of the substrate of b) displaying Z¹, or Z² as reactive groups with a solution comprising at least one type of functionalized hapten in an appropriate solvent, which is preferentially methanol, ethanol, water, methylene chloride, chloroform, dimethoxyethane, dioxane, tetrahydrofurane, diethyl ether, acetonitrile, dimethyl formamide, dimethylsulfoxide, in particular methanol, at an appropriate temperature.

In step a) a thin metal layer, preferably a thin gold-layer, is deposited on the substrate. This gold layer is preferably between 0.1 and 100 nm, in particular 1 and 10 nm thick. The substrate to be coated is preferably a polymer which can be chemically inert or not, in particular Topas™, polycarbonate, PMMA, glass or any other suitable material. The surface is covered with a thin metal layer or with a combination of metal layers, in particular with chromium and gold.

In step b) the appropriate reaction time for the solution comprising compounds with the formula (A) and/or with the formula (B) to be contacted with the metal-coated substrate is between several seconds to several days, preferably between 5 and 15 minutes. The appropriate reaction temperature in step b) and in step c) is between 0 and 50° C., but is preferentially room temperature.

The substrate produced by step b) may optionally be chemically modified by transient activation of the hydrophilic head groups Z¹ and/or Z². Example 4 discloses how these optional activations can be performed.

In step c) the appropriate solvent can be any solvent, but is particularly selected from the group consisting of methanol, ethanol, water, methylene chloride, chloroform, dimethoxyethane, dioxane, tetrahydrofurane, diethyl ether, acetonitrile, dimethyl formamide, dimethylsulfoxide, whereby methanol is most preferred.

The substrate surface resulting from the step b) displays the groups Z¹ or Z² which are defined above as reactive groups. These reactive groups can be reacted in step c) with the at least one type of functionalized hapten in an appropriate solvent. The reactions that take place at that time point strongly depend on the character of the group Z¹ or Z². These reactions in particular may involve a nucleophilic displacement by the adequately chemically reactive hapten of a nucleofugal substituent in Z¹ or Z² (example 1), a nucleophilic displacement by Z¹ or Z² of a nucleofugal substituent on the hapten molecule (example 2), or an addition to Z¹ or Z² of the adequately chemically reactive hapten (example 3):
Example 1 (Z²=—NH—C(O)—CH₂—Br, bromoacetamide):
Hapten-SH+Br—CH₂—C(O)NH—R->Hapten-S—CH₂—C(O)NH—R
Example 2 (Z²=—C(O)Cl, acid chloride):
Hapten-NH₂+Cl-C(O)—R->Hapten-NH—C(O)—R
Example 3 (Z²=—NC(O)CH═CH—C(O), maleimide):
Hapten-SH+(O)C—CH═CH—C(O)N—R->Hapten-S—CH—CH₂—C(O N(C(O))R.

For a more extensive description of relevant chemical reactions that take place in step c), one may refer to “Bioconjugate techniques”, G. T. Hermanson Ed., Academic Press, 1996 which is hereby incorporated by reference.

The invention also relates to a method for the quantitative detection of at least one type of hapten-specific biological macromolecule receptor in a test probe, in particular for the quantitative detection of at least one type of antibody in a test probe, comprising the steps a) to c) mentioned above which are followed by steps d) to f):
d) treatment of the hapten-linked substrate of step c) with
   a first solution comprising a defined amount of the at least one type of hapten-specific biological macromolecule receptor which is fluorescently labelled and with
   a second solution comprising a defined amount of the test probe comprising at least one type of hapten-specific biological macromolecule receptor which is non-labelled,
   wherein the treatment of the hapten-linked substrate of step c) with the first and the second solutions can be performed simultaneously or consecutively in any order,
   resulting in a substrate surface on which all covalently linked haptens are specifically bound by their corresponding biological macromolecular receptors, fluorescently labelled and not,
   wherein the ratio between bound fluorescently labelled or non-labelled receptors depends on the amount of biological macromolecule receptors in the original test probe,
e) optionally washing of the substrate surface of d),
f) measurement of the fluorescence of the substrate surface of step d) or of step e) and quantitative determination of the amount of biological macromolecule receptor in the test probe.

The test probe which contains the hapten-specific biological macromolecule receptor, in particular the antibody, to be measured can be any appropriate probe of a human or animal, preferably blood or other body liquids, tissue probes and cell extracts.

The invention additionally refers to a substrate with a surface that is linked with at least one type of hapten and that shows reduced unspecific interactions with circulating compounds that do not specifically bind to the surface-linked haptens which is produced by the steps a) to c).

The invention also relates to the use of a substrate as defined above for the quantitative detection of at least one type of hapten-specific biological macromolecule receptor in a test probe.

The term “hapten-specific biological macromolecule receptor” refers in the following to all kinds of macromolecules present in a test probe that can specifically bind to said hapten, in particular this term refers to an antibody that binds specifically to said hapten.

In another aspect, the present invention relates to a method for producing a substrate with a surface that is linked with at least one type of a biological macromolecule and that shows reduced unspecific interactions with circulating compounds that do not specifically bind to the surface-linked biological macromolecules, comprising the following steps:
a) production of a metal-coated substrate by deposition of a thin metal layer onto the surface of the substrate,
b) production of a substrate that is linked to locally perfluorinated thiols and/or disulfides displaying Z¹ and/or Z² as reactive groups by the treatment of the metal-coated substrate of a) with a solution comprising at least one compound selected from the group of locally perfluorinated thiols with the fomula (A)
   HS—Y¹[—(CX¹X²)ₙ—(CF₂)ₘ—(CX³X⁴)ₚ—]_{Q}Y¹ (A)
   which are defined as above mentioned
   and/or comprising at least one compound selected from the group of perfluorinated disulfides with the fomula (B) 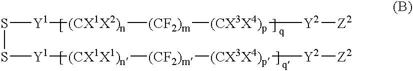
   which are defined as above mentioned
   in an appropriate solvent, which is preferentially methanol or ethanol, for an appropriate time and at an appropriate temperature,
c) production of a substrate that is covalently linked with at least one type of hapten by the treatment of the substrate of b) displaying Z¹, or Z² as reactive groups with a solution comprising at least one type of functionalized hapten in an appropriate solvent, which is preferentially methanol, ethanol, water, methylene chloride, chloroform, dimethoxyethane, dioxane, tetrahydrofurane, diethyl ether, acetonitrile, dimethyl formamide, dimethylsulfoxide, in particular methanol, at an appropriate temperature.

In step a) a thin metal layer, preferably a thin gold-layer, is deposited on the substrate. This gold layer is preferably between 0.1 and 100 nm, in particular 1 and 10 nm thick. The substrate to be coated is preferably a polymer which can be chemically inert or not, in particular Topas™, polycarbonate, PMMA, glass or any other suitable material. The surface is covered with a thin metal layer or with a combination of metal layers, in particular with chromium and gold.

In step b) the appropriate reaction time for the solution comprising compounds with the formula (A) and/or with the formula (B) to be contacted with the metal-coated substrate is between several seconds to several days, preferably between 5 and 15 minutes. The appropriate reaction temperature in step b) and in step c) is between 0 and 50° C., but is preferentially room temperature.

The substrate produced by step b) may optionally be chemically modified by transient activation of the hydrophilic head groups Z¹ and/or Z². Example 4 discloses how these optional activations can be performed.

In step c) the appropriate solvent can be any solvent, but is particularly selected from the group consisting of methanol, ethanol, water, methylene chloride, chloroform, dimethoxyethane, dioxane, tetrahydrofurane, diethyl ether, acetonitrile, dimethyl formamide, dimethylsulfoxide, whereby methanol is most preferred.

The substrate surface resulting from the step b) displays the groups Z¹ and/or Z² which are defined above as reactive groups. These reactive groups can be reacted in step c) with the at least one type of functionalized hapten in an appropriate solvent. The reactions that take place at that time point strongly depend on the character of the group Z¹ or Z². These reactions in particular may involve a nucleophilic displacement by the adequately chemically reactive hapten of a nucleofugal substituent in Z¹ or Z² (example 1), a nucleophilic displacement by Z¹ or Z² of a nucleofugal substituent on the hapten molecule (example 2), or an addition to Z¹ or Z² of the adequately chemically reactive hapten (example 3):
Example 1 (Z²═—NH—C(O)—CH₂—Br, bromoacetamide):
Hapten-SH+Br—CH₂—C(O)NH—R->Hapten-S—CH₂—C(O)NH—R
Example 2 (Z²═—C(O)Cl, acid chloride):
Hapten-NH₂+Cl—C(O)—R->Hapten-NH—C(O)—R
Example 3 (Z²═—NC(O)CH═CH—C(O), maleimide): Hapten-SH+(O)C—CH═CH—C(O)N—R->Hapten-S—CH—CH₂—C(O N(C(O))R.

For a more extensive description of relevant chemical reactions that take place in step c), one may refer to “Bioconjugate techniques”, G. T. Hermanson Ed., Academic Press, 1996 which is hereby incorporated by reference.

The invention also relates to a method for the quantitative detection of at least one type of hapten in a test probe comprising the steps a) to c) mentioned above which are followed by steps d) to f):
d) treatment of the macromolecule-linked substrate of step c) with
   a first solution comprising a defined amount of the at least one type of macromolecule-specific hapten which is fluorescently labelled and with a second solution comprising a defined amount of the test probe comprising at least one type of macromolecule-specific hapten which is non-lahelled, wherein the treatment of the macromolecule-linked substrate of step c) with the first and the second solution can be performed simultaneously or consecutively in any order,
   resulting in a substrate surface on which all covalently linked macromolecules are specifically bound by their corresponding haptens, fluorescently labelled and not,
   wherein the ratio between bound fluorescently labelled and non-labelled haptens depends on the amount of hapten in the original test probe,
e) optionally washing of the substrate surface of d),
f) measurement of the fluorescence of the substrate surface of step d) or of step e) and quantitative determination of the amount of haptens in the test probe.

The test probe which contains the hapten to be measured can be any appropriate probe of a human or animal, preferably blood or other body liquids, tissue probes and cell extracts.

The invention additionally refers to a substrate with a surface that is linked to at least one type of hapten-specific biological macromolecule receptor, which is preferentially a hapten-specific antibody, and that shows reduced unspecific interactions with circulating compounds that do not specifically bind to the surface-linked macromolecules, which is produced by the above steps a) to c).

The invention also relates to the use of a substrate as defined above for the quantitative detection of at least one type of hapten in a test probe.

The invention is further related to the use of a chemical compound with the fomula (A) or with the formula (B), which are defined as mentioned above, for the production of a substrate with a surface that is linked to at least one type of hapten and that shows reduced unspecific interactions with circulating compounds that do not specifically bind to the surface-linked haptens.

The invention is also related to the use of a chemical compound with the fomula (A) or with the formula (B), which are defined as mentioned above, for the production of a substrate with a surface that is linked to at least one type of a biological macromolecule, particularly with at least one type of antibody, and that shows reduced unspecific interactions with circulating compounds that do not specifically bind to the surface-linked biological macromolecules.

### EXAMPLE 1

General. ¹H—, ¹³C—, and ¹⁹F-NMR chemical shifts 8 are reported in ppm relative to their standard reference (1H: CHCl₃ at 7.27 ppm; ¹³C: CDCl₃ at 77.0 ppm; ¹⁹F: CFCl₃ external at 0.00 ppm). IR spectra were recorded in wave numbers (cm⁻¹). Mass Spectra (MS) were recorded at chemical ionization (CI) or in the electro spray (ES) mode. Mass data are reported in mass units (m/z). Abbreviations: s, singlet; d, doublet; t, triplet; q, quadruplet; m, multiplet; b, broad.

Compound 1(0).

Compound 8 (12 mg, 22 μmol) and potassium carbonate (12 mg, 87 μmol) are stirred in MeOH (2 ml) for 1 h. Saturated aqueous NH₄Cl is added and the resulting mixture is extracted with AcOEt. The organic layer is dried over MgSO₄ and reduced under vacuum to yield compound 1(0) (11 mg, 91%) as a slightly yellow solid.

TLC R_{f} 0.6 (CH₂Cl₂/AcOEt 95:5). ¹H-NMR (CDCl₃, 200 MHz) δ 4.09 (t, J=13.9 Hz, 2H); 4.00 (t, J=14.2 Hz, 2H); 3.76 (t, J=6.3 Hz, 2H); 2.73 (td, J=6.3, 8.3 Hz, 2H); 1.60 (t, J=8.3 Hz, SH).

Compound 1(1).

Compound 1(1) (37 mg, 90%) is prepared from 12 following the same procedure as described for 1(0).

TLC R_{f} 0.4 (Et₂O/hexanes 75:25). ¹H-NMR (CDCl₃, 200 MHz) δ 4.03 (tt, J=1.2, 13.8 Hz, 2H); 3.99 (tt, J=1.2, 13.8 Hz, 2H); 3.82-3.72 (m, 6H); 2.72 (td, J=6.3, 8.3 Hz, 2H); 1.60 (t, J=8.3 Hz, SH). ¹³C-NMR (CDCl₃, 75 MHz) δ 119.33-106.77 (m); 74.58; 74.26; 68.19 (t, J=25.3 Hz); 68.00 (t, J=25.3 Hz); 61.71; 24.10. ¹⁹F-NMR (CDCl₃, 188 MHz) 8-118.12 (m, 4F); −120.45 (m, 8F); −121.92 (m, 4F). IR (film) ν 3417 (b); 2930; 1204; 1146.

Compound 2(0).

Compound 8 (120 mg, 0.21 mmol) and sodium hydroxide (42 mg, 1.06 mmol) are stirred in MeOH/EtOH 1:1 (7 ml) for 1 h at room temperature. Then iodine (162 mg, 0.64 mmol) is added and the mixture is stirred for 1 h before saturated aqueous NH₄Cl is added. The solvent is removed under reduced pressure and the residue is extracted with AcOEt. The organic layer is dried over MgSO₄, and the solvent is removed to yield pure compound 2(O) (109 mg, 99%) as a slightly yellow solid.

TLC R_{f} 0.3 (CH₂Cl₂/AcOEt 95:5). ¹H-NMR (CDCl₃, 300 MHz) δ 4.00 (t, J=14.3 Hz, 4H); 3.97 (t, J=14.0 Hz, 4H); 3.86 (t, J=6.5 Hz, 4H); 2.89 (t, J=6.6 Hz, 4H). ¹³C-NMR (CDCl₃, 75 MHz) δ 119-107 (m); 71.05; 67.88 (t, J=25.5 Hz); 60.01 (t, J=25.3 Hz); 38.14. IR (film) ν 3325 (b); 2924; 1198; 1146.

Compound 2(1).

Compound 12 (141 mg, 0.23 mmol), sodium hydroxide (46 mg, 1.16 mmol), iodine (138 mg, 0.54 mmol), and water (50 μl) are stirred at room temperature in ethyl alcohol (7 ml) for 6 h. Saturated aqueous NH₄Cl is added, ethanol is removed under vacuum and the residue is extracted with AcOEt. The organic layer is washed with 2% aqueous Na₂S₂O₃, dried over MgSO₄, and reduced in vacuo to yield compound 2(1) (126 mg, 95%) as a slightly yellow solid.

TLC R_{f} 0.5 (CH₂Cl₂/AcOEt 75:25). ¹H-NMR (CDCl₃, 300 MHz) δ 4.02 (t, J=14.0 Hz, 4H); 3.99 (t, J=13.7 Hz, 4H); 3.90 (t, J=6.5 Hz, 4H); 3.79-3.73 (m, 8H); 2.91 (t, J=6.5 Hz, 4H). ¹³C-NMR (CDCl₃, 75 MHz) δ 119-107 (m); 74.27; 71.12; 68.16 (t, J=25.1 Hz); 67.96 (t, J=25.1 Hz); 61.66; 38.20. IR (film) ν 3392 (b); 2927; 1201; 1146.

Compound 2(4).

Compound 2(4) (49 mg, 99%) is obtained as a slightly yellow oil starting from compound 16 following the same procedure as described for the preparation of 2(1).

TLC R_{f} 0.3 (CH₂Cl₂/AcOEt 75:25). ¹H-NMR (CDCl₃, 200 MHz) δ 4.04 (t, J=14.2 Hz, 4H); 3.98 (t, J=14.6 Hz, 4H); 3.87 (t, J=6.6 Hz, 4H); 3.79-3.58 (m, 16H); 2.90 (t, J=6.3 Hz, 4H). ¹³C-NMR (CDCl₃, 75 MHz) δ 119-107 (m); 72.34; 72.10; 71.06; 70.22-70.15 (m); 69.98; 69.84; 68.01 (t, J=24.7 Hz); 67.9 (t, J=24.6 Hz); 61.46; 38.25. IR (film) ν 3440 (b); 2924; 2857; 1461; 1209; 1144.

Compound 3 (1).

A mixture of triphenylphosphine (32 mg, 124 mmol) and DIAD (25 μl, 176 μmol) in anhydrous THF (0.5 ml) is added to diol 2(1) (47 mg, 42 μmol) and maleimid (20 mg, 207 μmol) in THF (2 ml). The resulting solution is stirred for 2.5 h at room temperature, reduced under vacuum, and the residue is purified by silica gel chromatography (CH₂Cl₂AcOEt 9:1 to 0:10) to yield compound 3(1) (27 mg, 51%) as a slightly yellow solid.

¹H-NMR (CDCl₃, 300 MHz) δ 6.72 (s, 4H); 3.99 (t, J=14.0 Hz, 4H); 3.95 (t, J=14.1 Hz, 4H); 3.88 (t, J=6.6 Hz, 4H); 3.85-3.76 (m, 8H); 2.92 (t, J=6.6 Hz, 4H). ¹³C-NMR (CDCl₃, 75 MHz) δ 170.41; 134.14; 119-106 (m); 71.15; 69.39; 68.00 (t, J=23.1 Hz); 67.67 (t, J=26.1 Hz); 38.17; 36.95. IR (film) ν 3102; 2927; 1710; 1406; 1204; 1147.

Compound 4(1).

Bromoacetyl chloride (6.6 μl, 80 μmol) is added to a mixture of diol 2(1) (22 mg, 19 μmol) and triethylamine (11.2 μl, 80 μmol) in anhydrous THF (1 ml). The reaction mixture is stirred for 5 h at room temperature and volatiles are removed under vacuum. The residue is purified by silica gel chromatography (hexanes/AcOEt 8:2) to yield 4(1) (13 mg, 50%) as a slightly yellow oil.

¹H-NMR (CDCl₃, 200 MHz) δ 4.42-4.37 (m, 4H) 4.10 (s, 4H); 4.02 (t, J=14.0 Hz, 4H); 3.98 (t, J=13.9 Hz, 4H); 3.97-3.82 (m, 8H); 2.92 (t, J=6.6 Hz, 4H). ¹³C-NMR (CDCl₃, 50 MHz) δ 1167.20; 121-105 (m); 71.14; 70.42; 68.120 (t, J=25.6 Hz); 68.03 (t, J=25.6 Hz);64.71; 40.60; 38.17. IR (film) ν 2924; 2858; 1752; 1198; 1145.

Compound 5.

Sodium hydride (60% in oil, 0.23 g, 5.84 mmol) is added to 1,1,10,10-tetrahydroperfluorodecane-1,10-diol (2.25 g, 4.87 mmol) in anhydrous THF (40 ml) at 0° C. The mixture is stirred at room temperature for 2 h. Then HMPA (0.85 ml, 4.87 mmol) and tert-butyl bromoacetate (1.42 ml, 9.74 mmol) are added and the reaction mixture is stirred for 18 h at room temperature. Aqueous NH₄Cl is added and the solution is extracted with AcOEt. The organic layer is dried over MgSO₄, reduced under vacuum, and the residue is purified by silica gel chromatography (CH₂Cl₂/hexanes 50:50 to 100:0, then to CH₂Cl₂/AcOEt 90:10) to yield compound 5 (1.14 g, 41%) as a white solid.

TLC R_{f} 0.8 (CH₂Cl₂/AcOEt 90:10). ¹H-NMR (CDCl₃, 200 MHz) δ 4.12 (s, 2H); 4.09 (t, J=13.7 Hz, 2H); 4.07 (t, J=13.6 Hz, 2H); 1.49 (s, 9H). ¹³C-NMR (CDCl₃, 75 MHz) δ 168.68; 119-106 (m); 82.65; 69.66; 68.00 (t, J=25.0 Hz); 60.50 (t, J=25.1 Hz); 27.98. IR (film) ν 3468 (b); ν 2985; 2942; 1740; 1459; 1208; 1145.

Compound 6.

Method A:

Ethylene carbonate (236 mg, 2.67 mmol), 1,1,10,10-tetrahydroperfluorodecane-1,10-diol (412 mg, 0.89 mmol), and triethylamine (0.25 ml, 1.78 mmol) are vigorously stirred at 100° C. After a 16 h period, a second portion of ethylene carbonate (236 mg, 2.67 mmol) is added and the mixture is stirred for an additional 20 h period. The crude reaction mixture is directly purified by flash chromatography over silica gel (CH₂Cl₂/AcOEt 90:10 to 75:25) to yield compound 6 (168 mg, 36%).

Method B:

Lithium aluminum hydride (0.12 g, 3.06 mmol) is added by portion to compound 5 (1.17 g, 2.04 mmol) in diethyl ether (20 ml) at 0° C. The reaction mixture is stirred for 30 min before saturated aqueous Na₂SO₄ is added. The resulting solution is stirred for 5 min at 0° C. and dry Na₂SO₄ and sand are added to obtain a well dispersed suspension. The solid is removed by filtration, rinsed with diethyl ether and the organic layer is dried over MgSO₄, filtered and reduced in vacuo. The crude residue is purified by silica gel chromatography to yield compound 6 (1.01 g, 98%) as a white solid.

TLC R_{f} 0.4 (CH₂Cl₂/AcOEt 75:25). ¹H-NMR (CDCl₃, 300 MHz) δ 3.94 (t, J=14.0 Hz, 2H); 3.93 (t, J=14.7 Hz, 2H); 3.65 (m, 4H). ¹³C-NMR (CDCl₃, 75 MHz) δ 119-106 (m); 74.17; 67.92 (t, J=25.1 Hz); 61.07; 59.67 (t, J=25.1 Hz). IR (film) ν 3436 (b); 2924; 1802; 1769.

Compound 8.

Methanesulfonyl chloride (76 μl, 0.99 mmol) is added dropwise to a mixture of alcohol 6 (500 mg, 0.99 mmol) and triethylamine (140 μl, 0.99 mmol) in anhydrous THF (15 ml). The mixture is stirred for 75 min at room temperature. Diethyl ether (50 ml) is added and the resulting solution is washed with 5% HCl, and water. The organic layer is dried over MgSO₄ and reduced in vacuo to yield crude compound 7 (588 mg). Anhydrous DMF (5 ml) and potassium thioacetate (225 mg, 1.97 mmol) are added to the residue that is stirred at 80° C. for 90 min. Then water is added and the solution is extracted with AcOEt. The organic layer is washed with water, brine, dried over MgSO₄, and reduced under vacuum. The crude residue is purified by silica gel chromatography (CH₂Cl₂/AcOEt 95:5) to yield compound 8 (120 mg, 22%) as a slightly yellow solid.

TLC R_{f} 0.7 (CH₂Cl₂/AcOEt 95:5). ¹H-NMR (CDCl₃, 200 MHz) δ 4.08 (td, J=13.7, 6.1 Hz, 2H); 3.96 (t, J=6.4 Hz, 2H); 3.73 (t, J=6.3 Hz, 2H); 2;35 (s, 3H). ¹³C-NMR (CDCl₃, 50 MHz) δ 195.43; 119-106 (m); 71.53; 67.94 (t, J=25.5 Hz); 60.67 (t, J=25.7 Hz); 30.50; 28.54. IR (film) ν 3457; 2939; 2883; 1680.

Compound 9.

Sodium hydride (60% in oil, 617 mg, 15.4 mmol) is added to a mixture of 1,1,10,10-tetrahydroperfluoro-decane-1,10-diol (1.78 g, 3.86 mmol) and HMPA (1.34 ml, 7.72 mmol) in anhydrous THF (40 ml) at room temperature. The reaction mixture is stirred for 4 h before aqueous NH₄Cl is added, and extracted with AcOEt. The organic layer is dried over MgSO₄, reduced under vacuum and the residue is purified by silica gel chromatography (hexanes/CH₂Cl₂ 75:25 to 50:50) to yield compound 9 (1.52 g, 92%) as a white solid.

TLC R_{f} 0.5 (hexanes/CH₂Cl₂ 50:50). ¹H-NMR (CDCl₃, 300 MHz) δ 4.09 (s, 4H); 4.08 (t, J=12.8 Hz, 4H); 1.46 (s, 18H). ¹³C-NMR (CDCl₃, 50 MHz) δ 168.42; 121-109 (m); 82.33; 69.55; 67.94 (t, J=25.1 Hz); 27.92. IR (film) v 2983; 2938; 1747; 1373; 1211; 1144.

Compound 10.

Method A:

Compound 10 (146 mg, 30%) is obtained as a side product in the preparation of compound 6 following method A.

Method B:

Lithium aluminum hydride (244 mg, 6.43 mmol) is added to diester 9 (1.48 g, 2.14 mmol) in anhydrous diethyl oxide (25 ml) at 0° C. The mixture is stirred for 1 h before saturated aqueous Na₂SO₄ is added. After 10 min, solid Na₂SO₄ is added and the precipitate is removed by filtration. The filtrate is dried over MgSO₄ and reduced under vacuum to yield pure diol 10 (1.07 g, 91%) as a white solid.

TLC R_{f} 0.3 (hexanes/CH₂Cl₂ 50:50). ¹H-NMR (CDCl₃, 300 MHz) δ 4.03 (t, J=13.7 Hz, 4H); 3.77 (m, 8H). ¹³C-NMR (CDCl₃, 50 MHz) δ 119-106 (m); 74.28; 68.10 (t, J=25.0 Hz); 61.58. IR (film) ν 3382 (b); 2928; 1461; 1202; 1145.

Compound 12.

Methanesulfonyl chloride (40 μl, 524 Mmol) in anhydrous THF (3 ml) is added dropwise to diol 10 (288 mg, 524 μmol) and triethylamine (73 μl, 524 μmol) in THF (10 ml) at room temperature. The mixture is stirred for 2 h, diethyl ether (25 ml) is added and the solution is washed with HCl 2%. The aqueous phase is extracted with AcOEt. The organic layers are combined, dried over MgSO₄ and reduced under vacuum to yield a white crude residue (325 mg). Compound 11 is obtained as a mixture with diol 10 and its bismethanesulfonyl ester and is not separated. Anhydrous DMF (6 ml) and potassium thioacetate (117 mg, 1.02 mmol) are added. The mixture is stirred for 90 min at 85-90° C. and water is added. The mixture is extracted with AcOEt. The organic layer is washed with water, dried over MgSO₄, reduced under vacuum, and the residue is purified by silica gel chromatography (CH₂Cl₂/AcOEt 85:15) to yield compound 12 (140 mg, 44%) as a slightly yellow oil.

TLC R_{f} 0.3 (CH₂Cl₂/AcOEt 75:25). ¹H-NMR (CDCl₃, 300 MHz) δ 4.00 (t, J=14.0 Hz, 2H); 3.94 (t, J=13.7 Hz, 2H); 3.72-3.68 (m, 6H); 3.08 (t, J=6.2 Hz, 2H); 2.33 (s, 3H). ¹³C-NMR (CDCl₃, 50 MHz) δ 194.96; 119-106 (m); 74.24; 71.50; 68.18 (t, J=25.1 Hz); 67.90 (t, J=25.9 Hz); 61.67; 30.46; 29.50. IR (film) ν 3429 (b); 2940; 2880; 1694; 1204; 1146.

Compound 13.

Method A:

Sodium hydride (60% in oil, 100 mg, 2.5 mmol) is added to diol 10 (1.24 g, 2.25 mmol) in anhydrous THF (40 ml). The mixture is stirred for 90 min at 30-35° C. before HMPA (0.39 ml, 2.25 mmol) is added, followed by triethylene glycol trityl ether methanesulfonyl ester (1.06 g, 2.25 mmol) in one portion. The solution is refluxed for 16 h and saturated aqueous NH₄Cl is added. The mixture is extracted with AcOEt, the organic layer is dried over MgSO₄, reduced under vacuum and purified by silica gel chromatography (hexanes/AcOEt 70:30 to 25:75) to yield compound 13 (0.12 g, 6%) as a colorless oil.

Method B:

Lithium aluminum hydride (10 mg, 0.28 mmol) is added to t-butyl ester 18 (69 mg, 0.07 mmol) in anhydrous diethyl ether (10 ml) at 0° C. The reaction mixture is stirred for 1 h before saturated aqueous Na₂SO₄ (0.2 ml) is added. Solid Na₂SO₄ (1 g) is then added and the mixture is triturated, and filtered. The solid is washed twice with AcOEt, and the combined filtrate is reduced under vacuum to yield alcohol 13 (64 mg, 99%).

TLC R_{f} 0.5 (hexanes/AcOEt 50:50). ¹H-NMR (CDCl₃, 200 MHz) δ 7.50-7.46 (m, 6H); 7.34-7.19 (m, 9H); 4.01 (t, J=12.7 Hz, 4H); 3.77-3.67 (m, 18H); 3.25 (t, J=5.1 Hz, 2H). ¹³C-NMR (CDCl₃, 50 MHz) δ 144.07; 128.66; 127.69; 126.85; 119-106 (m); 86.48; 74.22; 72.23; 70.6 (m); 68.19 (t, J=24.7 Hz); 68.12 (t, J=24.7 Hz); 63.26; 61.60. IR (film) ν 3457 (b); 2923; 2877; 1450; 1210; 1145.

Compound 14.

Methanesulfonyl chloride (34 μl, 440 μmol) is added to compound 10 (110 mg, 200 μmol) and triethylamine (62 μl, 440 μmol) in THF (2 ml) at room temperature. The mixture is stirred for overnight, ethyl acetate (25 ml) is added and the solution is washed with saturated aqueous NH₄Cl. The organic layer is dried over MgSO₄ and reduced under vacuum to yield crude bis-mesylate compound 17 as a white solid.

¹H-NMR (CDCl₃, 300 MHz) δ 4.39 (t, J=4.3 Hz, 4H); 4.03 (t, J=13.8 Hz, 4H); 3.90 (t, J=4.3 Hz, 4H) 3.04 (s, 6H)

The later residue is dissolved in anhydrous THF (4 ml) and added in one portion to a mixture of triethylene glycol monotrityl ether (78 mg, 200 mmol) and sodium hydride (60% in oil, 22 mg) previously refluxed for 15 min in THF (3 ml). The resulting suspension is stirred at 60° C. for 6 h before saturated aqueous NH₄Cl is added. The mixture is extracted with AcOEt, the organic layer is dried over MgSO₄ and reduced under vacuum. The residue is chromatographed over silica gel (hexanes/AcOEt/CH₂Cl₂ 50:40:10 to 25:50:25) to yield 14 (59 mg, 28%) as a white solid.

TLC R_{f} 0.6 (hexanes/AcOEt/CH₂Cl₂ 25:50:25). ¹H-NMR (CDCl₃, 200 MHz) δ 7.55-7.38 (m, 6H); 7.35-7.17 (m, 9H); 4.40 (t, J=3.9 Hz, 2H); 4.04 (t, J=13.8 Hz, 4H); 3.90 (t, J=3.9 Hz, 2H); 3.82-3.54 (m, 14H); 3.25 (t, J=5.3 Hz, 2H); 3.05 (s, 3H).

Compound 15.

Method A:

Potassium thioacetate (31 mg, 0.27 mmol) and compound 14 (114 mg, 0.10 mmol) are stirred in anhydrous DMF (3 ml) at 80° C. for 2 h. Water is added and the mixture is extracted with AcOEt. The organic layer is washed with water, dried over MgSO₄, and reduced under vacuum to yield pure compound 15 (110 mg, 99%) as a colorless oil.

Method B:

Methanesulfonyl chloride (24 μl, 300 μmol) is added to compound 13 (123 mg, 133 μmol) and triethylamine (43 μl, 310 μmol) in THF (5 ml) at room temperature. The mixture is stirred for 2 h, diethyl ether (25 ml) is added and the solution is washed with HCl 2%. The aqueous phase is extracted with AcOEt. The organic layers are combined, dried over MgSO₄ and reduced under vacuum to yield a white crude residue (137 mg). Intermediate compound 14 is not purified. Anhydrous DMF (3 ml) and potassium thioacetate (31 mg, 266 μmol) are added. The mixture is stirred for 90 min at 85-90° C. and water is added. The mixture is extracted with AcOEt. The organic layer is washed with water, dried over MgSO₄, and reduced under vacuum to yield pure compound 15 (131 mg, 99%) as a slightly yellow oil. TLC R_{f} 0.3 (hexanes/AcOEt 70:30). ¹H-NMR (CDCl₃, 300 MHz) δ 7.47-7.43 (m, 6H); 7.30-7.18 (m, 9H); 3.99 (t, J=14.0 Hz, 2H); 3.95 (t, J=14.7 Hz, 2H); 3.75-3.64 (m, 16H); 3.22 (t, J=5.3 Hz, 2H); 3.09 (t, J=6.2 Hz, 2H); 2.33 (s, 3H). ¹³C-NMR (CDCl₃, 50 MHz) δ 195.27; 144.11; 128.70; 127.72; 126.89; 119-106 (m); 86.52; 72.28; 71.52; 70.65 (m); 68.25 (t, J=25.1 Hz); 67.92 (t, J=25.1 Hz); 63.30; 30.48; 28.52. IR (film) ν 2921; 2851; 1692; 1450.

Compound 16.

Compound 15 (130 mg, 132 μmol) is stirred in THFI/MeOH 1:2 (5 ml) with Amberlyst A15 (15 mg) at 60° C. for 2 h. The resin is removed by filtration, the filtrate is reduced under vacuum, and the residue is purified by silica gel chromatography (CH₂Cl₂/AcOEt 75:25 to 0:100) to yield compound 16 (52 mg, 53%) as a yellow oil.

TLC R_{f} 0.2 (CH₂Cl₂/AcOEt 75:25). ¹H-NMR (CDCl₃, 300 MHz) δ 4.03 (t, J=14.0 Hz, 2H); 3.96 (t, J=13.9 Hz, 2H); 3.80-3.58 (m, 18H); 3.10 (t, J=6.4 Hz, 2H); 2.34 (s, 3H).

¹³C-NMR (CDCl₃, 50 MHz) δ 195.27; 119-106 (m); 72.49; 72.30; 71.49; 70.65; 70.55; 70.47; 70.24; 68.24 (t, J=24.7 Hz); 67.90 (t, J=26.5 Hz); 61.65; 30.45; 28.49.

Compound 18.

Tributyl phosphine (158 μl, 0.63 mmol) is added dropwise to a mixture of compound 5 (104 mg, 0.18 mmol), triethylene glycol monotrityl ether (71 mg, 0.18 mmol), and N,N,N′,N′-tetramethyl azodicarboxamide (TMAD) (109 mg, 0.63 mmol) in refluxing anhydrous 1,4-dioxane (2 ml). The resulting solution is stirred for 90 min at 100° C. before the solvent is removed under vacuum. The crude residue is purified over silica gel (hexanes/AcOEt 80:20) to yield compound 18 (49 mg, 27%) as a colorless oil.

TLC R_{f} 0.3 (hexanes/AcOEt 75:25). ¹H-NMR (CDCl₃, 200 MHz) δ 7.53-7.43 (m, 6H); 7.37-7.18 (m, 9H); 4.13 (s, 2H); 4.11 (t, J=13.8 Hz, 2H); 4.01 (t, J=14.2 Hz, 2H); 3.81-3.60 (m, 14H); 3.25 (t, J=5.3 Hz, 2H); 1.50 (s, 9H). ¹³C-NMR (CDCl₃, 50 MHz) δ 168.42; 144.11; 128.69; 127.71; 126.88; 86.50; 82.39; 72.27; 70.64 (m); 69.60; 68.23 (t, J=24.7 Hz); 67.98 (t, J=24.7 Hz); 63.28; 28.01. IR (film) ν 2926; 2877; 1746; 1451; 1212; 1145.

### EXAMPLE 2

### Coating of Surfaces

The sample surface (Topas™, polycarbonate, PMMA, glass, or any other valuable material) is coated with a thin metal layer (preferentially Au, 0.1-100 nm thick), or a combination of metal layers, deposited using a sputter coater or any other valuable method. Then the sample is immersed into or exposed to a solution of a fluorinated compound or a mixture of fluorinated compounds (typically compounds 1(n)-4(n)) in an adequate solvent (MeOH, EtOH, CHCl₃, . . . depending on the type of material used) for a few seconds to a few hours. The sample is then rinsed with adequate solvent and dried.

### EXAMPLE 3

### Assay For Detection of Non Specific Interactions in the Presence of Biological Macromolecules

The title compounds have been evaluated for their ability to prevent protein precipitation and non specific binding to solid surfaces using the Surface Plasmon Resonance (SPR) technology. Bare Au chips (SIA chips, BIAcore AB, Uppsala, Sweden) were treated with compounds 2(O), 2(1), and PEO₆-disulfide ([S(C₂H₄O)₆—OH]₂) (0.1 mM in ethanol for 5 minutes, washed with ethanol, then water) and mounted in the BIAcore apparatus.

They were studied in parallel with dextran coated chips (CM5 chip, BLAcore AB) for comparison. The different chips were treated with reconstituted standard human plasma (Dade Behring Marburg GmbH, Marburg, Germany; Lot. No. 502577; albumin concentration: 75 mg/mL) diluted (1/10) in Hepes buffer pH 7.4. Uncorrected non specific binding of the plasma proteins to the substrates was quantified subtracting the initial resonance signal expressed in resonance units (RU) (recorded before introduction of the diluted serum into the flow cell) from that recorded after 1 minute exposition to the plasma solution followed by 10 seconds washing with Hepes buffer. The results are reported in Table 1. The corrected values account for the thickness of each substrate and the average distance (d) separating the immobilized proteins from the gold layer since the BIAcore weighs the mass of a bound ligand by a factor that decays exponentially the longer the distance of the ligand from the sensor surface.

The results obtained are reported in table 1.

| | | Resonance units | |
|---|---|---|---|
| Coating | Thickness (nm) | Uncorrected | Corrected |
| Dextran CM5 | 100 | 241 | 430 |
| PEO₄₅ | | | |
| 2(0) | 2.0 | 556 | 558 |
| 2(1) | 2.3 | 421 | 425 |
| PEO₆ | | | |
| 2(4) | 2.3 | 880 | 889 |

The results indicate that the claimed fluorinated compounds show about the same resonance signal as dextran. However, due to the large differences when considering the thickness of the coatings, the PEO₆disulfide coating was investigated in addition. Polyethylene oxides (PEO) are known to efficiently prevent protein adsorption on surfaces. PEO₆disulfide and compound 2(1), both of similar length, were investigated in terms of resonance signals. Both products result in layers of similar thickness on gold. Therefore, the SPR signal may be reproducibly interpreted in a comparable manner. In this experimental setup, results clearly demonstrate that fluorinated compounds exhibit very good performances in preventing protein adsorption on the BIAcore chip surface and reduce non specific protein adsorption by more than a two-fold factor when compared with a standard coating of equal thickness. These results show that the background noise signal is markedly reduced. The interpretation of the data, however, should consider that using SPR as detection technology, the closer the ligand will bound to the gold layer the stronger the corresponding signal amplitude since the amplitude of the evanescent electric field is maxium close to the gold layer.

### EXAMPLE 4

### Applications of the Invention

The substrates provided by the present invention can be applied for example to the manufacture of functionalized chips for bioassays.

The substrate to be coated is preferably a polymer which can be chemically inert or not, in particular PreTopas™, polycarbonate, PMMA, glass or any other suitable material. The surface is covered with a thin metal layer or with a combination of metal layers, in particular with chromium and gold. The metal layer reacts with a locally perfluorinated thiol or disulfide, or a mixture of locally perfluorinated thiols and/or disulfides which had been adequately functionalized. Thiols and disulfides react with gold to form Au-S bonds.

Thiol compounds of formula (A) are more readily available by synthesis than disulfides of formula (B) which are usually prepared starting from a thio]precursor. However the SH-functional group in (A) is not compatible with some Z² groups, as for instance maleimide, bromoacetamide, acid chloride, depending on experimental conditions, such as solvent, pH, temperature. Disulfides which can result from the oxidation of thiol precursors exhibit about the same reactivity toward gold layers as parent thiols and are compatible with these Z² groups in a wider range of experimental conditions. Therefore it may be necessary to use a (mixture of) locally perfluorinated disulfide(s) instead of thiol(s).

The resulting coating can subsequently be chemically modified by transient activation of the hydrophilic head groups Z¹ and/or Z² or not. Whether an activation or chemical modification of these hydrophilic head groups is necessary depends on the nature of the hydrophilic groups Z¹ and/or Z² and on the nucleophily/electrophily of the reactive hapten.

In the following, examples of Z¹ and/or Z² are given that do require activation for coupling to a reactive hapten or macromolecule:

1—Examples of Z Groups that do not Require Activation for Coupling to a Reactive Hapten Molecule.

If hapten is nucleophilic:

—C(O)—ONC(O)C₂H₄(O)
—NHC(O)CH₂X
—C(O)—NC(O)CH═CHC(O) —C(O)X
—OP(O)X₂—CH═CHCH₂X
—C(O)OP(O)(OMe)₂—N═C═S
—N═C=O, where X represents halogen
   * If hapten is electrophilic:
—OH
—NH₂
—SH
   2—Examples of Z groups that do not require activation for coupling to a biological macromolecule.
—C(O)—ONCLO)C₂H₄(O)
—NHC(O)CH₂X
—C(O)—NC(O)CH═CHC(O) —C(O)X
—OP(O)X₂—CH═CHCH₂X
—C(O)OP(O)(OMe)₂—N═C═S
—N═C═O, where X represents halogen
   3—Examples of Z groups that may require intermediate activation/transformation for coupling to a hapten.
—OH
—OP(O)(OH)₂—C(O)OH
—S—C(O)CH₃
   4—Examples of Z groups that may require intermediate activation/transformation for coupling to a biological macromolecule.
—OH
—OP(O)(OH)₂
—C(O)OH

### COUPLING EXAMPLES

### EXAMPLE 4.1

A substrate of (1)b with at least one compound with Z²═NC(O)CH═CHC(O) can be treated with a thiol derivative of digoxin. Digoxin is a molecule incorporating a steroid moiety and is often used alone or in combination with other therapeutics in the treatment of chronic heart failure. Digoxin coupled to the reactive surface is capable of binding circulating anti-digoxin antibodies that may have been raised in patients treated with digoxin thereby lowering its efficacy and compromising the therapeutic outcome. The need for a sensitive and specific detection of these antibodies explains the clinical importance of the present invention that may be used in manufacturing of a biochip for a specific assay in routine practice.

### EXAMPLE 4.2

A substrate of (2)b with at least one compound with Z═NC(O)CH═CHC(O) can be treated with reduced (i.e. displaying SH groups) anti-protein C antibody fragment. Its immobilization permits the detection and quantification of protein C target analyte in the sample material examined by immunological methods. This will allow for the detection of alterations in protein C concentration during anticoagulation monitoring in routine practice.

### EXAMPLE 5

### Applications of the Invention

The present invention is of particular interest when measurement of target analytes in various fluids, e,g, biological fluids or waste effluents, may be exposed to interfering substances such as non target macromolecules that may bind non specifically to the reactive surface thereby reducing sensitivity and/or specificity of the measured signal.

The locally perfluorinated compounds described herein may be used in various solid phase assay formats which include but are not limited to homogeneous or heterogeneous immunoassays, competitive or non competitive or immunochromatographic detection methods, surface plasmon resonance (SPR) based assay.

Examples include concentration measurements of protein C in whole venous blood, troponin I in plasma, immunoglobulin G₁ in spinal cord liquor and tetracyclines in agricultural effluents. All analytes are measured individually by competitive immunoassay in microfluidic cartridges precoated with the fluorinated compounds within minutes of applying a sample volume ranging from 1 to 15 μL of the respective fluid. The labeled analyte present at saturating concentrations competes with the unlabeled target present in the sample added. The concentration ranges of analyte that may be determined and the respective molecular weight are shown in the Table below:

| | Sample | | |
|---|---|---|---|
| Target Analyte | Material | Concentration Range | Molecular Weight |
| Protein C | Whole Blood | 0.001-20 μg/mL | 62 kD |
| Troponin I | Plasma | 0.01-50 ng/mL | 22 kD |
| Tetracyclines | Agricultural | 0.01-100 μg/mL | 0.4 kD |
| | Effluent | | |
| Immuno- | Liquor | 0.05-15 mg/mL | 150 kD |
| globulin | | | |

Low detection level capabilities and the lowest possible false positive rates are essential requirements for protein C (coagulation disorders), troponin I (myocardial damage), tetracyclines (potential effluent contamination) and immunoglobulin G, (cerebrospinal infections) measurement in routine practice.

In the following the invention is described in detail by FIGS. 1 to 3 and Scheme 1 to 6.

FIG. 1: General architecture of the perfluorinated compounds used for the generation of substrate surfaces that are covalently linked to haptens or biological macromolecules showing reduced non-specific interactions: The central linear perfluorinated chain (black segment) prevents hydrophilic and hydrophobic interactions; an “anchor” segment (grey segment) is designed to interact tightly with the support; a hydrophilic head group (striped segment) is designed for good wettability and further chemical reactivity.

FIG. 2: Competitive, homogenous immuno assay with fluorescence detection after surface functionalization with a hapten.

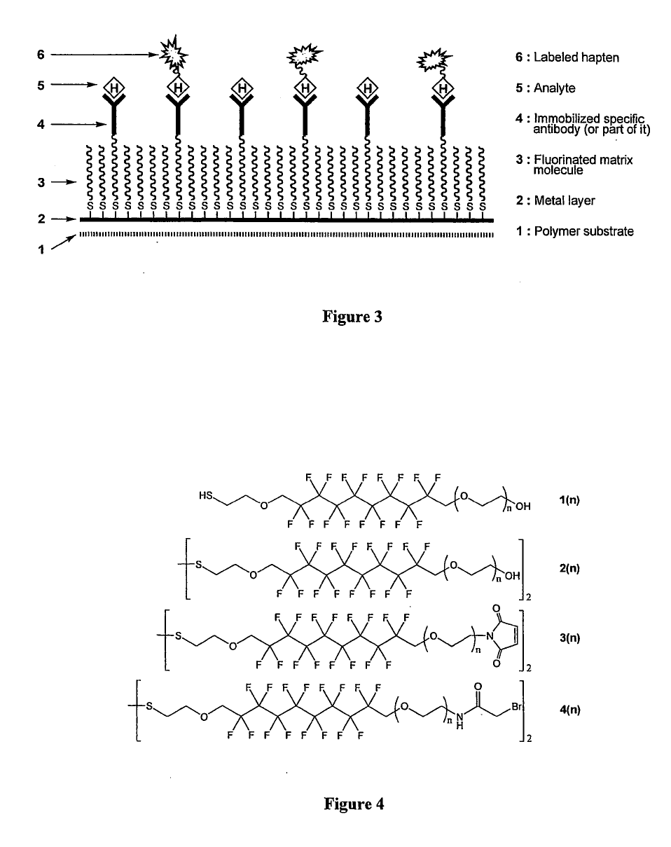

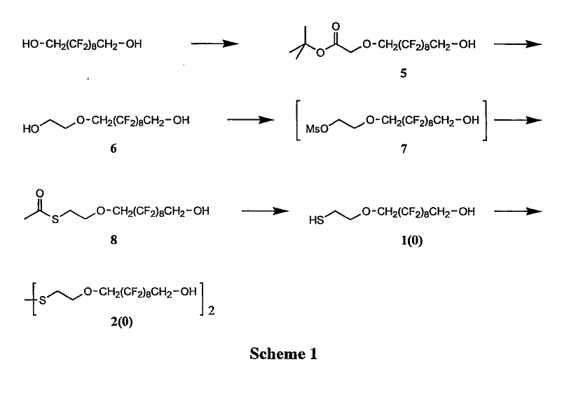

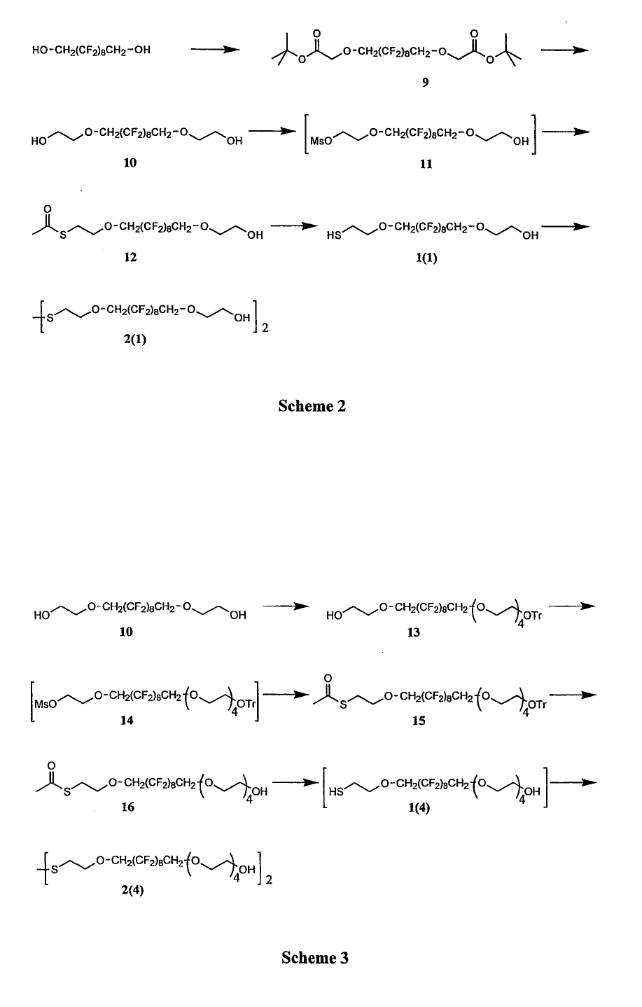

FIG. 3: Competitive, homogenous immuno assay with fluorescence detection after surface functionalization with an antibody.

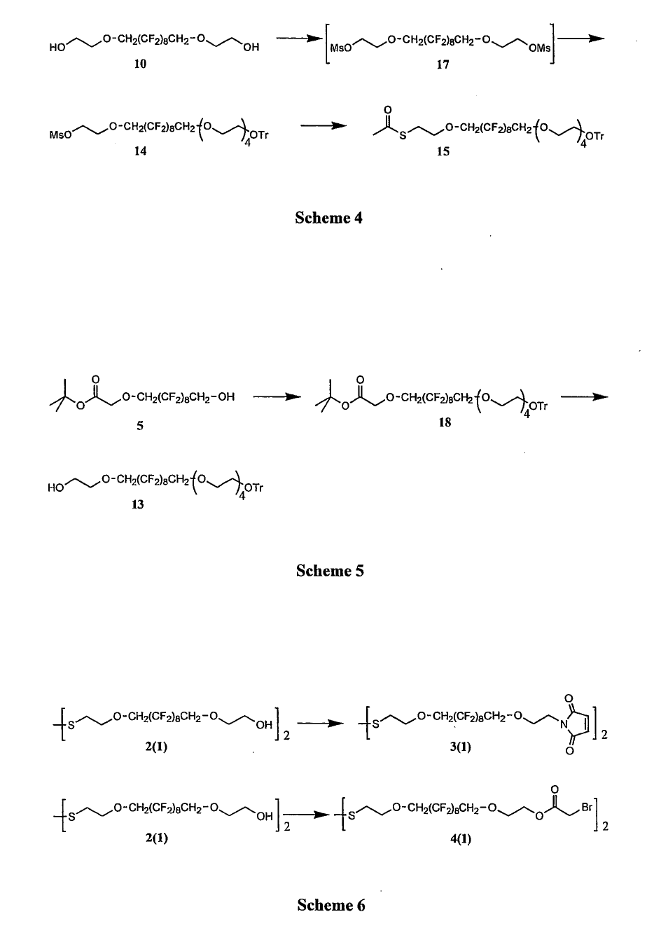

The different molecules involved in the preparation of the above-mentioned substrates are described in FIG. 4. Their preparation has been achieved according to schemes 1-6.

1. A chemical compound with the fomula (A)
(A) HS—Y¹[(—CX¹X²)ₙ(—CF₂)ₘ(—CX³X⁴)ₚ—]_{q}Y²—Z¹ (A)
wherein each
X¹, X², X³ and X⁴ is independently from each other selected from the group consisting of
hydrogen, halogen, an alkyl group optionally substituted by one or several halogens, an acyl group optionally substituted by one or several halogens, a hydrocarbon group incorporating one or several double bonds optionally substituted by one or several halogens, an aralkyl group optionally substituted by one or several halogens, an aryl group optionally substituted by one or several halogens, a hydrocarbon group containing one or several heteroatoms that is optionally unsaturated;
wherein each
Y¹ and Y² is independently from each other selected from the group consisting of
an alkylene group optionally containing one or several heteroatoms, an alkylene group that is optionally unsaturated, an alkylene group containing one or several heteroatoms that is optionally unsaturated;
wherein each
Z¹ is selected from the group consisting of
hydrogen, halogen, a group selected from AR¹, C(B¹)(AR¹), NR¹R², ASO₂R¹, ASO₂(AR¹), SOᵣR¹, SO₂(NR¹R²), AP(B²)(AR¹)(AR²), AP(B²)(AR¹)R², P(B²)(AR¹)(AR²), P(B²)(AR¹)R², P(B²)R¹R²
wherein each A is independently from each other selected from O, S or NR¹;
each B¹ is independently from each other selected from O, S or NR¹;
each B² is selected from O, S or Se;
each R¹ and R² is selected from
hydrogen, an alkyl group optionally substituted by one or several halogens, an acyl group, an acyl group optionally substituted by one or several halogens, a hydrocarbon group incorporating one or several double bonds optionally substituted by one or several halogens, an aralkyl group optionally substituted by one ore several halogens, an aryl group optionally substituted by one or several halogens, a hydrocarbon group containing one or several heteroatoms that is optionally unsaturated, an ammonium group, an ammonium group that is substituted by at least one hydrocarbon group, an ion M⁺_{1/N} in which M represents a metal and v is the valence state of metal M, an internal cation;
and wherein
n and p independently from each other represent an integer between 0 and 4;
m represents an integer between 1 and 22;
q represents an integer between 1 and 100.

2. A chemical compound with the fomula (A) as claimed in claim 1, wherein
n and p represent an integer having the value 0;
m represents an integer between 4 and 22;
q represents an integer having the value 1;
Y¹ is selected from the group consisting of —CH₂[—O—C₂H₄₁t with t representing an integer between 0 and 10;
Y² is selected from the group consisting of —CH₂—[O—C₂H₄—]ᵤ with u representing an integer between 0 and 100.

3. A chemical compound with the fomula (B) 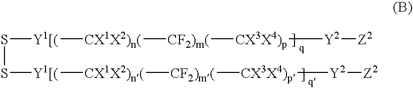
wherein each
X¹, X², X³ and X⁴ is independently from each other selected from the group consisting of
hydrogen, halogen, an alkyl group optionally substituted by one or several halogens, an acyl group optionally substituted by one or several halogens, a hydrocarbon group optionally incorporating one or several double bonds, a hydrocarbon group substituted by one or several halogens and incorporating one or several double bonds, an aralkyl group optionally substituted by one or several halogens, an aryl group optionally substituted by one or several halogens, a hydrocarbon group containing one or several heteroatoms that is optionally unsaturated;
wherein each
Y¹ and Y² is independently from each other selected from the group consisting of
an alkylene group optionally containing one or several heteroatoms, an alkylene group that is optionally unsaturated, an alkylene group containing one or several heteroatoms that is optionally unsaturated;
wherein each
Z² is independently from each other selected from the group consisting of hydrogen, halogen, a group selected from
AR¹, C(B¹)(AR¹), NR¹R¹, ASO₂R², ASO₂(AR¹), SOrR², SO₂(NR¹R¹), AP(B²)(AR¹)(AR¹), AP(B²)(AR¹)R², P(B²)(AR¹)(AR¹), P(B²)(AR¹)R², P(2)R²R², C²)R², C(B²)(B²R³), a N-maleimidyl group, an isocyanate group, an isothiocyanate group, A-C(═NR¹)R², O—NHR¹;
wherein
each A is independently from each other selected from the group consisting of O, S or NR¹;
wherein
each B¹ is independently from each other selected from the group consisting of O, S or NR¹;
wherein
each B² is independently from each other selected from the group consisting of O, S or Se;
wherein
each R¹ is independently from each other selected from the group consisting of
hydrogen, an alkyl group optionally substituted by one or several halogens, an acyl group optionally substituted by one or several halogens, a hydrocarbon group incorporating ne or several double bonds optionally substituted by one or several halogen atoms, an aralkyl group optionally substituted by one ore several halogen atoms, an aryl group optionally substituted by one or several halogen atoms, a hydrocarbon group containing one or several heteroatoms that is optionally unsaturated, an ammomiun group, an ion of formula M⁺_{1/V} in which M represents a metal and v is the valence state of metal M, an internal cation;
wherein
each R² is independently from each other selected from the group consisting of
hydrogen, halogen, a N₃ group, an alkyl group optionally substituted by one or several halogens, an acyl group optionally substituted by one or several halogens, a hydrocarbon group incorporating one or several double bonds, a hydrocarbon group substituted by one or several halogen atoms optionally incorporating one or several double bonds, an aralkyl group optionally substituted by one or several halogens, an aryl group optionally substituted by one or several halogens, a hydrocarbon group containing one or several heteroatoms that is optionally unsaturated, NR¹—NHR¹;
wherein
each R³ represents an acyl group optionally substituted by one or several halogens, SOₛR⁴ with s representing an integer between 0 and 2, P(B²)(A⁴)(AR⁴), P(B²)(AR⁴)R⁴, P(B²)R⁴RW, C(═NR¹)(NHR¹), C═N—R⁵—NR¹, a cyclic or acyclic imide group, in particular cyclic: NC(O)R⁵C(O), or acyclic: N(C(O)R⁴)(C(O)R⁴)];
wherein
each R⁴ is independently from each other selected from the group consisting of
an alkyl group optionally substituted by one or several halogens, a hydrocarbon group incorporating one or several double bonds that is optionally substituted by one or several halogens, an aralkyl group optionally substituted by one ore several halogens, an aryl group optionally substituted by one or several halogens, a hydrocarbon group containing one or several heteroatoms that is optionally unsaturated;
wherein
each R⁵ is independently from each other selected from the group consisting of
an alkylene group that optionally contains one or several heteroatoms, an alkylene group that is optionally unsaturated, a alkylene group containing one or several heteroatoms that is optionally unsaturated, an aryl diradical optionally containing one or several heteroatoms;
wherein
each n, n′, p, and p′ independently from each other represents an integer between 0 and 4;
each m and m′ independently from each other represent an integer an integer between 1 and 22;
each q and q′ independently from each other represent an integer between 1 and 100.

4. A chemical compound with the fomula (B) as claimed in claim 3, wherein
n, n′, p and p′ represent an integer of 0,
m and m′ represent an integer between 4 and 22
q and q′ represent an integer of 1
each Y¹ is —CH₂[—O—C₂H₄—]ₜ
each Y² is selected from the group consisting of CH₂[—O—C₂H₄—]ᵤNC(O)CH═CHC(O), —CH₂[O—C₂H₄—]ₜNHC(O CH₂Br;
with t representing an integer between 0 and 10 and
with u representing an integer between 0 and 100.

5. A method for manufacturing a substrate with a surface that is linked to at least one type of hapten and that shows reduced unspecific interactions with circulating compounds that do not specifically bind to the surface-linked haptens, comprising the following steps:
a) producing a metal-coated substrate by depositing of a thin metal layer onto the surface of the substrate,
b) producing a substrate that is linked to locally perfluorinated compounds by metal-sulfide bonds displaying Z¹ and/or Z² as reactive groups
by treating the metal-coated substrate of step a) with a solution comprising at least one compound selected from the group consisting of locally perfluorinated thiols with the fomula (A)
HS—Y¹[(—CX¹X²)ₙ(—CF₂)ₘ(—CX³X⁴)ₚ—]_{q}Y²—Z¹ (A)
as defined in claim 1 or 2,
and/or comprising at least one compound selected from the group consisting of locally perfluorinated disulfides with the formula (B) 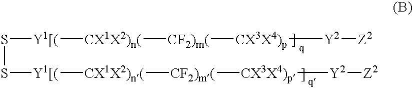
as defined in claim 3 or 4,
in an appropriate solvent for an appropriate time and at an appropriate temperature,
c) producing a substrate that is covalently linked with at least one type of hapten by contacting the substrate of b), displaying Z¹, or Z² as reactive groups, with a solution comprising at least one type of functionalized hapten in an appropriate solvent at an appropriate temperature.

6. The method for the quantitative detection of at least one type of hapten-specific biological macromolecule receptor in a test probe comprising the steps a) to c) as claimed in claim 5, followed by the following steps d) to f):
d) treating the hapten-linked substrate of step c) with a first solution comprising a defined amount of the at least one type of hapten-specific biological macromolecule receptor which is fluorescently labelled and with a second solution comprising a defined amount of the test probe comprising at least one type of hapten-specific biological macromolecule receptor which is non-labelled,
wherein the treatment of the hapten-linked substrate of step c) with the first and the second solutions can be performed simultaneously or consecutively in any order,
resulting in a substrate surface on which all covalently linked haptens are specifically bound by their corresponding biological macromolecular receptors, fluorescently labelled and not,
wherein the ratio between bound fluorescently labelled or non-labelled receptors depends on the amount of biological macromolecule receptors in the original test probe,
e) optionally washing of the substrate surface of d),
f) measuring the fluorescence of the substrate surface of step d) or of step e) and quantitative determination of the amount of biological macromolecule receptor in the test probe.

7. The method according to claim 6, wherein the at least one type of hapten-specific biological macromolecule receptor in the test probe is an antibody that binds specifically to the surface-linked hapten.

8. The method according to claim 6 or 7, wherein the test probe is a body liquid or extract selected from the group consisting of blood, tissue probes, cell extracts and waste effluents.

9. A substrate with a surface that is linked with at least one type of hapten and that shows reduced unspecific interactions with circulating compounds that do not specifically bind to the surface-linked haptens, which is produced by the steps a) to c) as claimed in claim 5.

10. The use of a substrate which is produced by the steps a) to c) as claimed in claim 5 for the quantitative detection of at least one type of hapten-specific biological macromolecule receptor in a test probe.

11. The use as claimed in claim 10, wherein the hapten-specific biological macromolecule receptor is an antibody.

12. A method for manufacturing a substrate with a surface that is linked to at least one type of a biological macromolecule and that-shows reduced unspecific interactions with circulating compounds that do not specifically bind to the surface-linked biological macromolecules, comprising the following steps:
a) producing a metal-coated substrate by depositing a thin metal layer onto the surface of the substrate,
b) producing a substrate that is linked to locally perfluorinated thiols and/or disulfides Z2 displaying Z¹ and/or Z² as reactive groups by treating the metal-coated substrate of a) with a solution comprising at least one compound selected from the group of locally perfluorinated thiols with the fomula (A)
HS—Y¹[(—CX¹X²)ₙ(—CF₂)ₘ(—CX³X⁴)ₚ—]_{q}Y²—Z¹ (A)
as defined in claim 1 or 2
and/or comprising at least one compound selected from the group of perfluorinated disulfides with the fomula (13) 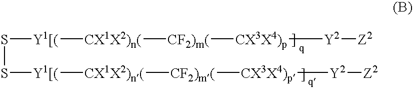
as defined in claim 3 or 4,
in an appropriate solvent for an appropriate time and at an appropriate temperature,
c) producing a substrate that is covalently linked to at least one type of biological macromolecule by contacting the substrate of step b) displaying Z or Z₂ as reactive groups with a solution comprising at least one type of chemically reactive biological macromolecule in an appropriate solvent and at an appropriate temperature.

13. The method as claimed in claim 12, wherein the at least one type of a biological macromolecule is an antibody.

14. Method for the quantitative detection of at least one type of hapten in a test probe comprising the steps a) to c) as claimed in claim 12 or 13, followed by the following steps d) to f):
d) treating the macromolecule-linked substrate of step c) with a first solution comprising a defined amount of the at least one type of macromolecule-specific hapten which is fluorescently labelled and with a second solution comprising a defined amount of the test probe comprising at least one type of macromolecule-specific hapten which is non-labelled,
wherein the treatment of the macromolecule-linked substrate of step c) with the first and the second solution can be performed simultaneously or consecutively in any order,
resulting in a substrate surface on which all covalently linked macromolecules are specifically bound to their corresponding haptens, fluorescently labelled and not,
wherein the ratio between bound fluorescently labelled and non-labelled haptens depends on the amount of hapten in the original test probe,
e) optionally washing of the substrate surface of d),
f) measuring the fluorescence of the substrate surface of step d) or of step e) and quantitative determination of the amount of haptens in the test probe.

15. The method according to claim 14, wherein the test probe is a body liquid or extract selected from the group consisting of blood, tissue probes, cell extracts and waste effluents.

16. A substrate with a surface that is linked to at least one type of a biological macromolecule and that shows reduced unspecific interactions with circulating compounds that do not specifically bind to the surface-linked biological macromolecules, which is produced by the steps a) to c) as claimed in claim 12.

17. A substrate according to claim 16, wherein the at least one type of a biological macromolecule is an antibody.

18. The use of a substrate which is produced by the steps a) to c) as claimed in claim 12 for the quantitative detection of at least one type of hapten in a test probe.

19. The use of a chemical compound with the fomula (A) as claimed in claim 1 or 2 and/or of a chemical compound with the formula (B) as claimed in claim 3 or 4 for the production of a substrate with a surface, that is linked to at least one type of hapten and that shows reduced unspecific interactions with circulating compounds that do not specifically bind to the surface-linked haptens.

20. The use of a chemical compound with the fomula (A) as claimed in claim 1 or 2 and/or of a chemical compound with the formula (B) as claimed in claim 3 or 4 for the production of a substrate with a surface, that is linked to at least one type of a biological macromolecule and that shows reduced unspecific interactions with circulating compounds that do not specifically bind to the surface-linked biological macromolecules.

21. The use as claimed in claim 20, wherein the at least one type of a biological macromolecule is an antibody.
